(19) United States
(12) Patent Application Publication
(10) Pub. No.: US 2026/0041447 A1
(43) Pub. Date: Feb. 12, 2026

(54) DISTAL STABILIZER

(71) Applicant: Bolt Medical Inc., Tokyo (JP)

(72) Inventor: Naoki INUZUKA, Tokyo (JP)

(73) Assignee: Bolt Medical Inc., Tokyo (JP)

(21) Appl. No.: 18/995,736

(22) PCT Filed: Jul. 19, 2023
(86) PCT No.: PCT/JP2023/026405

§ 371 Date: Jan. 16, 2025

(30) Foreign Application Priority Data
Jul. 21, 2022 (JP) 2022-116311

(51) Int. Cl. A61B 17/221; A61B 17/22

(52) U.S. Cl. CPC A61B 17/221; A61B 2017/22079; A61B 2017/2215

(57) ABSTRACT

Provided is a distal stabilizer that can mitigate the scattering of foreign matter from a locking stent to the distal side. A distal stabilizer 1 is used for catheter delivery in a body lumen, and includes: a linear delivery member 3; and a self-expanding member 2 that is linked to the distal end of the linear delivery member 3, and that has a cylindrical body section 11 locked inside the body lumen. The body section 11 includes a filter unit 17 constituted of a wire member 14 that protrudes towards the center in a radial direction RD from the circumferential side.

### TECHNICAL FIELD

The present invention relates to a distal stabilizer to be locked in a biological lumen.

### BACKGROUND ART

In a biological lumen such as an artery of a patient, a treatment using a treatment device or a treatment using a catheter itself as a treatment device is performed by delivering the treatment device to a target position using a lumen inside the catheter whose distal end is guided to the vicinity of the target position. For example, Patent Document 1 discloses a distal stabilizer (an anchoring device) in which a locking stent for anchoring is joined to the distal end of a delivery wire. When the locking stent is released from a microcatheter and expanded, since the locking stent is anchored to the inner wall in the biological lumen, the operation of delivering the treatment catheter that is slidably fitted over the microcatheter to the vicinity of the target position can be easily performed.

### CITATION LIST

### Patent Document

Patent Document 1: U.S. Pat. No. 968,221

### DISCLOSURE OF THE INVENTION

### Problems to be Solved by the Invention

As a use mode of the distal stabilizer, after anchoring the locking stent on the distal side of the thrombus, an aspiration catheter (treatment catheter) is delivered to the position of the thrombus, and the thrombus is aspirated by the aspiration catheter to be collected, or the thrombus is aspirated by the aspiration catheter and drawn to the proximal side to be collected. In such a use mode, the operation of delivering the aspiration catheter to the target location may cause the thrombus or fragments thereof to scatter on the distal side, causing embolization of the biological lumen on the distal side. Such embolization of the biological lumen on the distal side is not only caused by thrombus and fragments thereof, and may also be caused by, for example, plaque or the like accumulated on a blood vessel wall. Hereinafter, a thrombus or fragments thereof, plaque and the like that may cause embolization of a biological lumen will be collectively referred to as “foreign matter”.

An object of the present invention is to provide a distal stabilizer capable of suppressing scattering of foreign matter from a locking stent to a distal side.

### Means for Solving the Problems

The present invention is directed to a distal stabilizer for use in catheter delivery in a biological lumen, the distal stabilizer including: a linear delivery member; and a self-expansion member that is coupled to a distal end of the linear delivery member and includes a cylindrical main body portion to be locked in the biological lumen, in which the main body portion includes a filter portion constituted by a wire-shaped member that protrudes from an outer circumferential side toward a center side in a radial direction of the main body portion.

The filter portion may include a plurality of filter portions provided in an axial direction of the main body portion.

The plurality of filter portions each may include a plurality of wire-shaped members that each protrudes from the outer circumferential side toward the center side in the radial direction of the main body portion, and positions of the plurality of wire-shaped members in a circumferential direction may be different from one another,

In each of the plurality of filter portions, end portions of the plurality of wire-shaped members that each protrude from the outer circumferential side toward the center side in the radial direction of the main body portion may be coupled to each other.

The main body portion may have a mesh pattern structure in which a plurality of cells each having a shape surrounded by the plurality of wire-shaped members are spread, at least one of the plurality of cells may be an open cell having at. least one free protruding end, and at least one of the plurality of filter portions may be provided by the plurality of wire-shaped member constituting the free protruding end of the filter portion protruding from the outer circumferential side toward the center side in the radial direction of the main body portion.

The free protruding end of the cell may protrude toward a distal side in a direction of inserting the self-expansion member into a catheter.

The self-expansion member may be locked on a distal side of a target position in a biological lumen.

### Effects of the Invention

According to the present invention, it is possible to provide a distal stabilizer capable of suppressing scattering of foreign matter from a locking stent to a distal side.

### BRIEF DESCRIPTION OF THE DRAWINGS

FIG. 1 is a diagram illustrating an overall configuration of a delivery system 10 including a distal stabilizer 1 according to an embodiment;

FIG. 2 is a side view of a locking stent 2;

FIG. 3 is an expanded view showing a state in which the locking stent 2 is virtually expanded in a plane;

FIG. 4 is an expanded view in which only a filter portion 17 of FIG. 3 is drawn by a solid line;

FIG. 5A is a cross-sectional view taken along the line s1-s1 in FIG. 2;

FIG. 5B is a cross-sectional view taken along the line s2-s2 in FIG. 2;

FIG. 5C is a cross-sectional view taken along the line s3-s3 in FIG. 2;

FIG. 5D is a view of the locking stent 2 shown in FIG. 2 as viewed from the arrow a1;

FIG. 6 is a schematic diagram illustrating an example of a surgical operation performed by the delivery system 10 including the distal stabilizer 1;

FIG. 7 is a schematic diagram illustrating an example of a surgical operation performed by the delivery system 10 including the distal stabilizer 1;

FIG. 8 is a schematic diagram illustrating an example of a surgical operation performed by the delivery system 10 including the distal stabilizer 1;

FIG. 9 is a schematic diagram illustrating an example of a surgical operation performed by the delivery system 10 including the distal stabilizer 1;

FIG. 10 is a schematic diagram illustrating an example of a surgical operation performed by the delivery system 10 including the distal stabilizer 1;

FIG. 11 is a schematic diagram illustrating an example of a surgical operation performed by the delivery system 10 including the distal stabilizer 1;

FIG. 12 is a schematic diagram illustrating an example of a surgical operation performed by the delivery system 10 including the distal stabilizer 1;

FIG. 13 is an expanded view showing a state in which a locking stent 2A of a modification is virtually expanded in a plane; and

FIG. 14 is a side view of a locking stent 2B in a modification.

### PREFERRED MODE FOR CARRYING OUT THE INVENTION

Hereinafter, embodiments of a distal stabilizer according to the present invention will be described. It should be noted that all of the drawings attached to the present disclosure are schematic diagrams, and the shape, the scale, the vertical and horizontal dimensional ratio, and the like of each part are changed or exaggerated from actual ones in consideration of ease of understanding and the like. In this disclosure, etc., terms specifying a shape, a geometric condition, and a degree thereof, for example, a term such as “direction” includes a range generally regarded as the direction in addition to the strict meaning of the term. In addition, in the present disclosure, the long axis direction in a state where the distal stabilizer extends linearly is also referred to as an “axial direction LD” or simply as an “axial direction”. In the axial direction LD, the proximal side close to the practitioner is referred to as “D1”, and the distal side away from the practitioner is referred to as “D2”.

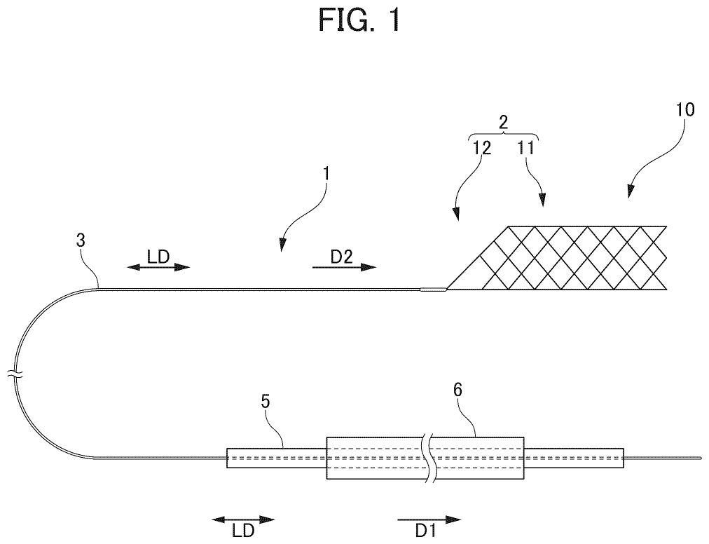

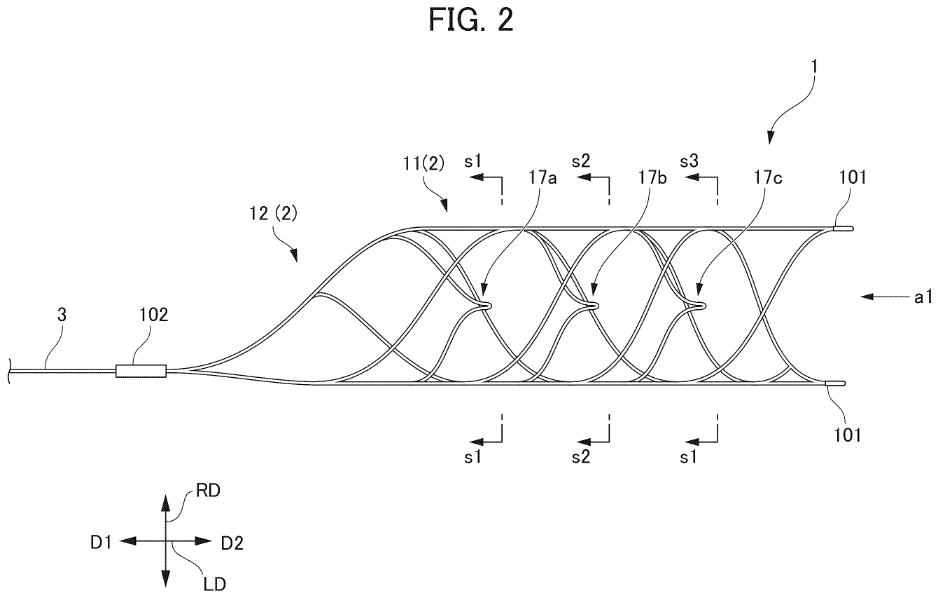

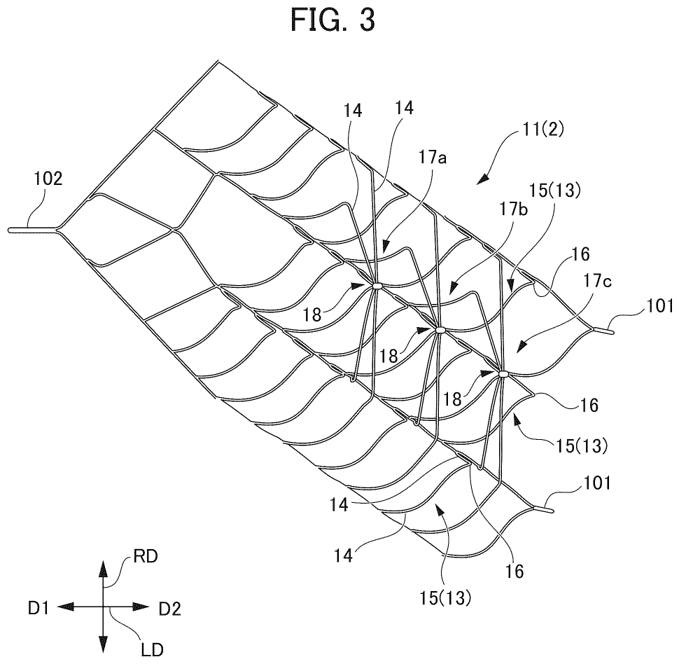

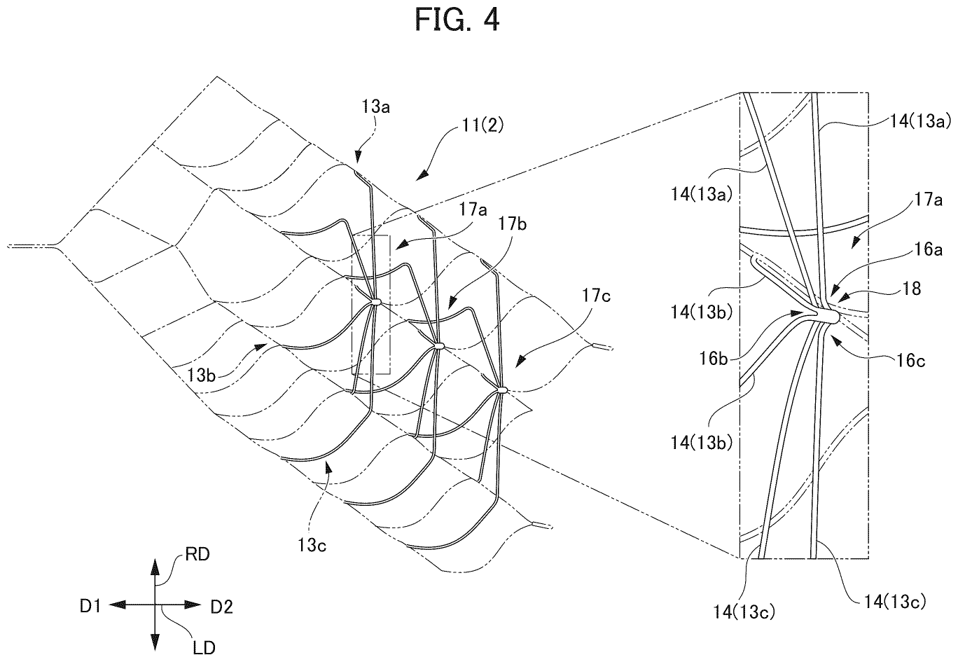

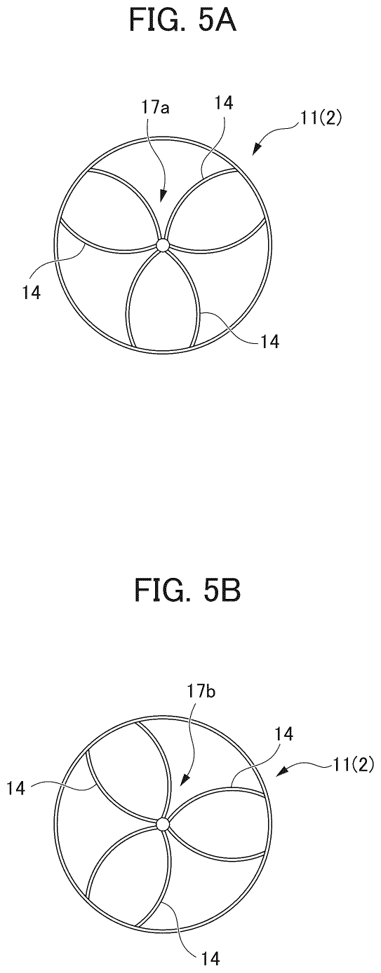

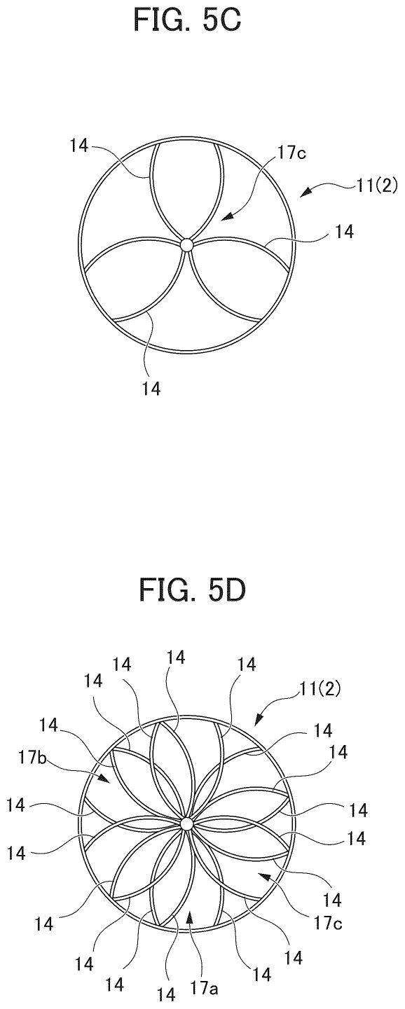

FIG. 1 is a diagram illustrating an overall configuration of a delivery system 10 including a distal stabilizer 1 according to an embodiment. FIG. 2 is a side view of a locking stent 2. FIG. 2 shows a state in which the locking stent 2 is expanded in diameter. FIG. 3 is an expanded view showing a state in which the locking stent 2 is virtually expanded in a plane. FIG. 4 is an expanded view in which only a filter portion 17 of FIG. 3 is drawn by a solid line. FIG. 5A is a cross-sectional view taken along the line s1-s1 in FIG. 2 FIG. 5B is a cross-sectional view taken along the line s2-s2 in FIG. 2. FIG. 5C is a cross-sectional view taken along the line s3-s3 in FIG. 2. FIG. 5D is a view of the locking stent 2 shown in FIG. 2 as viewed from an arrow a1.

The delivery system 10 shown in FIG. 1 is, for example, a system used to deliver a treatment device into a biological lumen. The blood vessels of the biological lumen in which the delivery system 10 is used are not particularly limited, and may be, for example, blood vessels (arteries and veins) of the brain, coronary, upper and lower limbs, organs, and the like. In the following description, the biological lumen is also referred to as a “blood vessel”. As shown in FIG. 1, the delivery system 10 includes a distal stabilizer 1 and a plurality of catheters, including a first catheter 5 and a second catheter 6. The distal stabilizer 1 is a device used for catheter delivery in a biological lumen. The distal stabilizer 1 includes a locking stent (self-expanding member) 2 and a delivery wire (linear delivery member) 3.

The locking stent 2 is an anchoring device that is inserted into the first catheter 5 in a state reduced in diameter and is locked to an inner wall V1 of the biological lumen V by being released from the first catheter 5 and expanding in the blood vessel. The locking stent 2 is coupled to the distal end of the delivery wire 3. As shown in FIG. 1, the locking stent 2 includes a main body portion 11 and an antenna portion 12. The main body portion 11 has a cylindrical shape and has a mesh pattern structure described later. In FIG. 1, the configuration of the main body portion 11 is simplified. As shown in FIG. 2, three filter portions 17a to 17c (hereinafter, collectively referred to as “filter portion 17”) are provided in the main body portion 11 along the axial direction LD. The antenna portion 12 is a portion that converges the portion on the proximal side D1 of the main body portion 11 to the delivery wire 3.

As shown in FIG. 2, distal markers 101 are provided at the end of the distal side D2 of the locking stent 2. At the end of the proximal side D1 of the locking stent 2, a proximal marker 102 is provided. Each marker is made of an X-ray opaque material. Each of the distal markers 101 is a portion serving as a mark for confirming the position of the end portion on the distal side D2 of the locking stent 2. The proximal marker 102 is a portion serving as a mark for confirming the position of the end on the proximal side D1 of the locking stent 2.

The locking stent 2 can be made, for example, by performing laser processing on a tube made of a biocompatible material, particularly preferably a superelastic alloy. In a case of producing the locking stent 2 from a superelastic alloy tube, it is preferable to produce the locking stent 2 by performing laser processing on the tube of about 2 to 3 mm, then expanding the tube to a desired diameter, and subjecting the tube to shape memory treatment. The locking stent 2 is not limited to laser processing, and may be produced by cutting or the like, or may be produced by braiding a wire-shaped metal wire into a tubular shape.

The locking stent 2 is preferably made of a material having high rigidity and high biocompatibility. Examples of such a material include titanium, nickel, stainless steel, platinum, gold, silver, copper, iron, chromium, cobalt, aluminum, molybdenum, manganese, tantalum, tungsten, niobium, magnesium, calcium, and alloys containing these. Further, as such a material, for example, a synthetic resin material of a polyolefin such as polyethylene (PE) or polypropylene (PP), polyamide, polyvinyl chloride, polyphenylene sulfide, polycarbonate, polyether, polymethyl methacrylate, or the like may be used. Further, as such a material, for example, a biodegradable resin (biodegradable polymer) such as polylactic acid (PLA), polyhydroxybutyrate (PHB), polyglycolic acid (PGA), or poly ε-caprolactone may be used.

Among these, titanium, nickel, stainless steel, platinum, gold, silver, copper, magnesium, and alloys containing these are preferable. Examples of the alloy include a Ni—Ti alloy, a Cu—Mn alloy, a Cu—Cd alloy, a Co—Cr alloy, a Cu—Al—Mn alloy, an Au—Cd—Ag alloy, and a Ti—Al—V alloy. Further, examples of the alloy include an alloy of magnesium and Zr, Y, Ti, Ta, Nd, Nb, Zn, Ca, Al, Li, Mn, or the like. Among these alloys, a Ni—Ti alloy is preferable.

As shown in FIG. 3, in the main body portion 11, cells including a plurality of open cell portions (described later) are spread in the circumferential direction. The circumferential direction is a direction along the outer circumference of the main body portion 11. The radial direction RD shown in FIGS. 3 and 4 corresponds to the circumferential direction in the main body portion 11 virtually expanded in a plane. In the main body portion 11, the plurality of cells 13 spread in the circumferential direction are continuously arranged along the axial direction. That is, the main body portion 11 has a mesh pattern structure in which the plurality of cells 13 are spread in the circumferential direction and are continuous in the axial direction. Each of the plurality of cells 13 is also referred to as an opening or a compartment, and refers to a portion surrounded by a wire-shaped strut (wire-shaped member) 14. Open cell portions 15 shown in FIG. 3 are cells each having a free protruding end 16 among the cells 13. In the present embodiment, all the cells 13 constituting the main body portion 11 are open cell portions 15.

In the locking stent 2 (main body portion 11) of the present embodiment, the cells 13 constituting the filter portion 17 (described later) are all open cell portions 15, but the other cells may be closed cells. That is, when the closed cells each including the free end is defined as an open cell and the closed cells each not including the free end is defined as a closed cell, the closed cells and the open cells may coexist in the cell pattern of the locking stent 2.

The free protruding end 16 is a portion where ends of two adjacent struts 14 are coupled in one cell 13, and is an end where another strut 14 is not connected to this portion. Here, the term “coupled” indicates not only a form in which they are integrally connected without a seam, but also a form in which they are connected by welding, UV bonding, infiltration of silver brazing, or the like. Each of the free protruding ends 16 protrudes toward the distal side D2 in the direction of inserting the locking stent 2 into the catheter. Further, each of the protruding forms of the free protruding ends 16 may be, for example, a substantially U-shape or a substantially Ω-shape, in addition to a substantially V-shape, as shown in FIG. 3. The ends of the two struts 14 constituting each of the free protruding ends 16 of the cells 13 forming the filter portion 17 (described later) are coupled to the ends of the two struts 14 constituting each of the free protruding ends 16 of the other cells 13 forming the filter portion 17.

As described later, the filter portion 17 is a portion that collects foreign matter such as thrombus or fragments thereof when the foreign matter migrates toward the distal side D2 by the operation of delivering the second catheter 6 to the target position TP. As shown in FIG. 3, the filter portion 17 is provided by the two struts 14 constituting each of the free protruding ends 16 of the cells 13 protruding from the outer circumferential side to the center side in the radial direction RD. The filter portion 17 has a basket shape in which the struts 14 and the free protruding ends 16 protrude toward the distal side D2 in a state in which the locking stent 2 is expanded in diameter.

Next, the configuration of the filter portion 17 will be described by taking a filter portion 17a disposed on the most proximal side D1 of the locking stent 2 as an example. As shown in FIG. 4, in the filter portion 17a, in three cells 13a, 13b, and 13c arranged in the circumferential direction (corresponding to the radial direction RD in FIG. 4), the end portions of the respective struts constituting the respective free protruding ends 16a to 16c are coupled to each other.

That is, in the cell 13a, as a member constituting the filter portion 17a, two struts 14a constituting the free protruding end 16a protrude from the outer circumferential side to the center side in the radial direction RD. In the cell 13b, as a member constituting the filter portion 17a, two struts 14b constituting the free protruding end 16b protrude from the outer circumferential side to the center side in the radial direction RD. In the cell 13c, as a member constituting the filter portion 17a, two struts 14c constituting the free protruding end 16c protrude from the outer circumferential side to the center side in the radial direction RD. In the cells 13a to 13c, the ends of the respective struts constituting the filter portion 17a are coupled to each other to constitute one tip portion 18. The tip portion 18 protrudes to the distal side D2. The other filter portions 17b and 17c shown in FIG. 4 are configured in the same manner.

When a general stent is virtually expanded, it has a planar shape without a protruding portion as a whole. On the contrary, as shown in FIGS. 3 and 4, when the main body portion 11 of the locking stent 2 according to the present embodiment is virtually expanded, the filter portion 17a has a three-dimensional shape protruding toward the center side in the radial direction RD more than the other portions.

As shown in FIG. 4, the filter portions 17a to 17c are spaced apart from each other in the axial direction LD at substantially equal intervals. Further, the struts 14 constituting the filter portions 17a to 17c are provided at different positions in the circumferential direction (corresponding to the radial direction RD in FIG. 4). Therefore, when the main body portion 11, which is expanded as shown in FIG. 3, is returned to the cylindrical shape, the positions of the struts 14 constituting the filter portions 17a to 17c in the circumferential direction are different from one another as shown in FIGS. 5A to 5C. As a result, as shown in FIG. 5D, when the locking stent 2 is viewed from the axial direction LD, each of the struts 14 constituting the filter portions 17a to 17c are substantially evenly arranged (or not extremely unevenly arranged). The plurality of cells 13 (a part of each of which protrudes toward the center) constituting the filter portion 17 are in a relationship positioned between the plurality of cells 13 constituting the other filter portions 17 along the arrow al (see FIG. 2). That is, the positions in the circumferential direction of the plurality of cells 13 constituting the filter portions 17 are shifted from the positions in the circumferential direction of the plurality of cells 13 constituting the other filter portions 17. Therefore, the filtering function by the plurality of filter portions 17 is further improved.

FIGS. 5A to 5D schematically illustrate shapes and arrangements of the respective struts 14 constituting the filter portions 17a to 17c. The function and operation of the locking stent 2 having the above-described filter portions 17a to 17c will be described later.

Returning to FIG. 1, the delivery wire 3 is a member used when the locking stent 2 is advanced or retracted in the biological lumen. The delivery wire 3 is delivered toward the distal side D2 when the locking stent 2 is advanced within the biological lumen, and is drawn toward the proximal side D1 when the locking stent 2 is retracted within the biological lumen V. The delivery wire 3 is made of, for example, a metal material having a high elastic modulus such as stainless steel. In addition, the diameter of the delivery wire 3 is not particularly limited as long as it has physical properties sufficient to perform an advancing or retracting operation in the biological lumen and is adapted to the first catheter 5, and may be, for example, 0.005 to 0.018 inches. The linear delivery member is not limited to a metal material such as a delivery wire, and may be made of, for example, a resin or a Composite material of a metal and a resin.

As shown in FIG. 1, the first catheter 5 is slidably fitted over the delivery wire 3. The first catheter 5 is, for example, a microcatheter. The second catheter 6 (described later) is slidably fitted over the first catheter 5. The diameter of the first catheter 5 is set according to a target position, and the inner diameter and the degree of bending of a biological lumen of the path to the target position TP, and is not particularly limited, but the inner diameter is preferably 0.017 inches or less, and more preferably 0.0165 inches or less. As the catheter, one or more other catheters (not shown) slidably fitted over the second catheter 6 may be used as necessary. In general, a large number of catheters can be used to ultimately insert and advance a large inner diameter catheter into a biological lumen.

Among the plurality of catheters including the second catheter 6, a catheter having an inner diameter larger than that of the first catheter 5 is referred to as a treatment catheter. The treatment catheter is a catheter having an inner diameter sufficient to place the treatment device thereinside in an inserted manner or having an inner diameter sufficient to use itself as a treatment device. The treatment catheter may also be referred to as a guiding catheter in applications that place the treatment device thereinside in an inserted manner. Examples of the treatment device include a thrombus aspiration device, a flow diverter, an aneurysm embolization device, a thrombectomy device (such as a stent retriever), a stent for treating aneurysm, a stent for treating intracranial arterial stenosis, a balloon catheter, a shunt, and a liquid embolic substance release means (such as a catheter having a lumen through which a liquid embolic substance passes). The treatment catheter may itself be used as a treatment device. In such applications, the treatment catheter may also be referred to as a thrombus aspiration catheter. In the embodiment described below, a case where the second catheter 6 is a thrombus aspiration catheter (treatment catheter) will be described as an example.

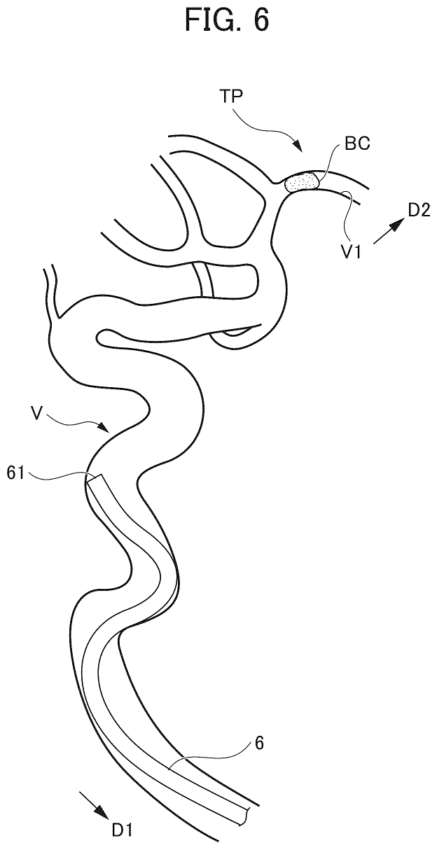

Next, a mode of use of the delivery system 10 including the distal stabilizer 1 will be described. FIGS. 6 to 12 are schematic diagrams each illustrating one example of a surgical operation by the delivery system 10 including the distal stabilizer 1. Here, a surgical operation of collecting a thrombus formed at a target position in a biological lumen will be described.

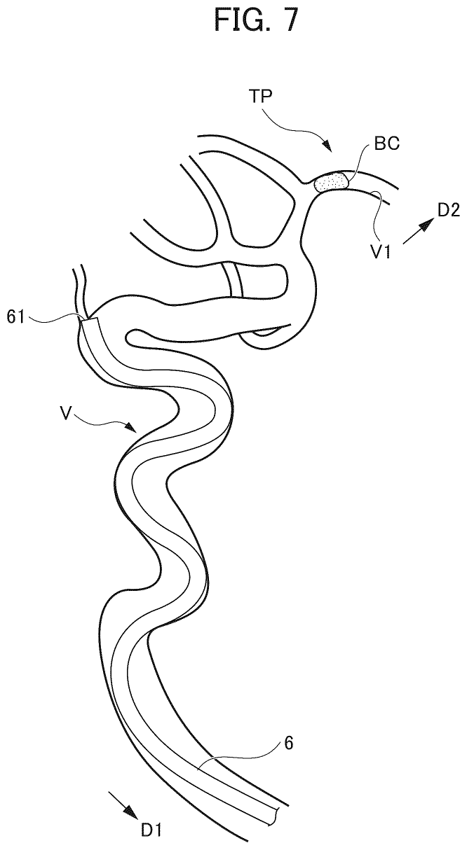

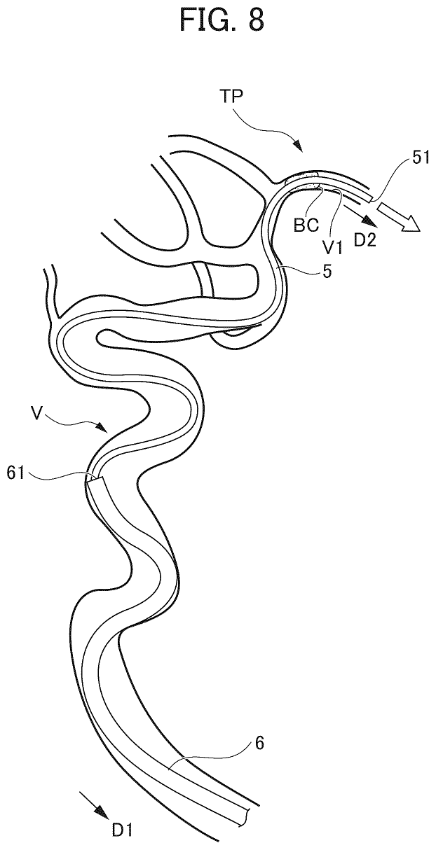

First, the second catheter 6 is disposed on the proximal side D1 of the biological lumen V of a patient. Typically, as shown in FIGS. 6 and 7, a distal end 61 of the second catheter 6 is caught by the bending portion or the branching portion of the biological lumen V, and it is difficult to advance the second catheter 6 further to the distal side. As shown in FIG. 8, the first catheter 5 is inserted into the second catheter 6 to be delivered into the biological lumen V and pushed out of the distal end 61 of the second catheter 6, and then a distal end 51 of the first catheter 5 is disposed at the target position TP. A thrombus BC is formed at the target position TP. The distal end 51 of the first catheter 5 is advanced through the thrombus BC to reach the distal side D2.

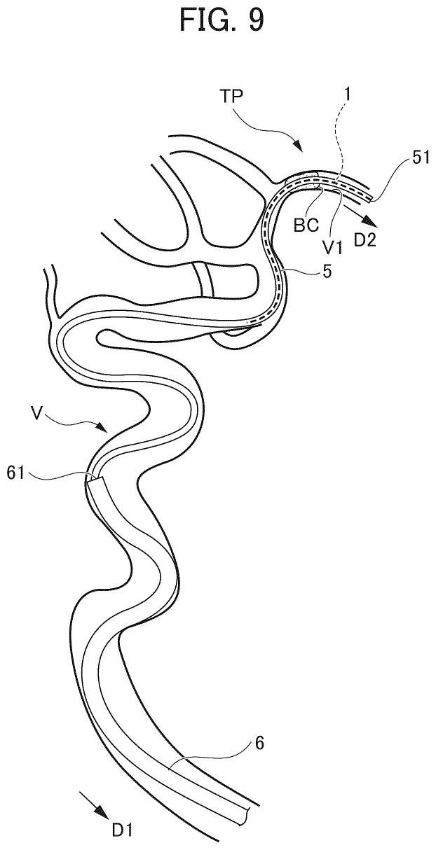

Subsequently, as shown in FIG. 9, the distal stabilizer 1 is inserted into the first catheter 5 and delivered to the target position TP. At this time, the locking stent 2 of the distal stabilizer 1 is accommodated in the first catheter 5 in a state reduced in diameter. In FIG. 9, the distal stabilizer 1 (the locking stent 2 and the delivery wire 3) is also accommodated on the proximal side of the first catheter 5, but for convenience, the distal stabilizer 1 is illustrated by a broken line only on the distal side of the first catheter 5.

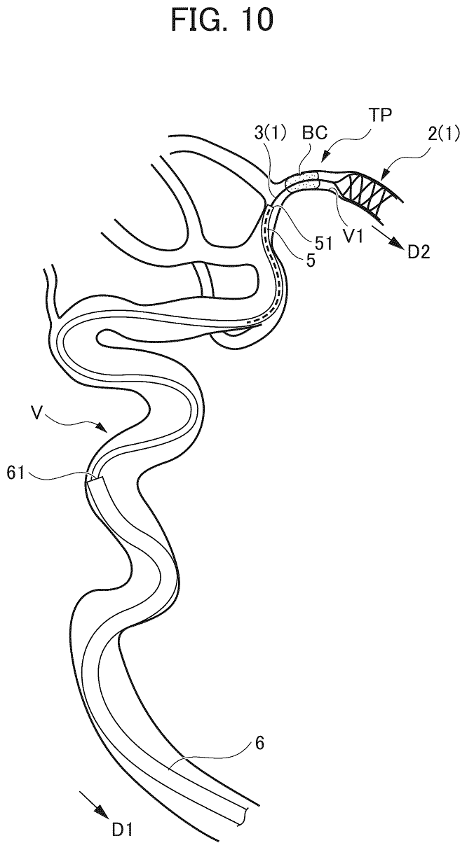

Next, as shown in FIG. 10, the locking stent 2 accommodated in the first catheter 5 in the state reduced in diameter is released from the distal end 51 of the first catheter 5. Release of the locking stent 2 is performed by an operation of retracting the first catheter 5 toward the proximal side D1. The locking stent 2 released from the distal end 51 of the first catheter 5 expands in diameter due to its self-expanding force. As a result, the locking stent 2 pushes the inner wall V1 of the biological lumen V from the inside toward the outside, and is locked to the inner wall V1. The locking stent 2 (the main body portion 11) is locked to the distal side of the target position TP where the thrombus BC is formed in the biological lumen V. Since the locking stent 2 has the open cell portion 15 from each of which the free protruding end 16 (refer to FIG. 3) protrudes, the locking stent 2 can be locked to the inner wall V1 with a strong locking force.

When the locking stent 2 expands in diameter, the filter portion 17 provided inside the locking stent 2 also expands in the radial direction to form a basket shape having the filter portion 17 expanded inside as shown in FIG. 2. As a result, even when foreign matter such as a thrombus BC or fragments thereof migrate to the distal side D2 by the operation of delivering the second catheter 6 to the target position TP described later, most of the foreign matter can be collected by the filter portions 17 of the locking stent 2. In FIG. 10, the filter portions 17 of the locking stent 2 are not shown.

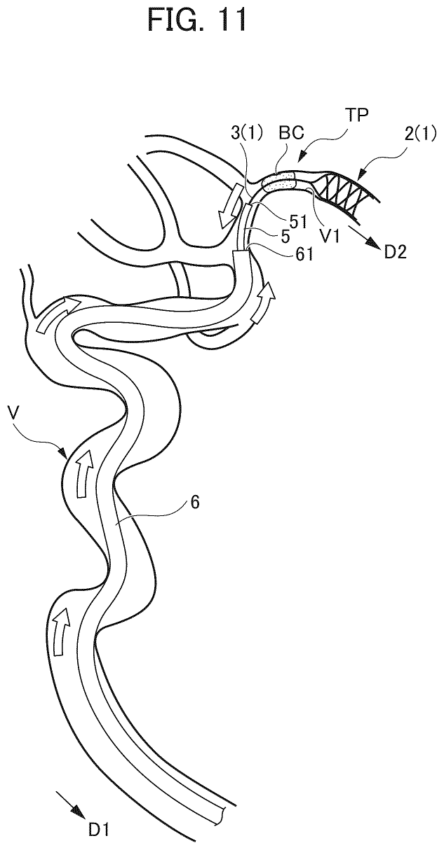

Subsequently, as shown in FIG. 11, the second catheter is slidably fitted over the first catheter 5, and the second catheter 6 is advanced to the distal side D2 in the biological lumen V. The second catheter 6 has a large outer diameter and high rigidity. Therefore, the second catheter 6 repeatedly applies a force to pull the first catheter 5 or the delivery wire 3 having low rigidity toward the proximal side D1 in the process of advancing. When it is difficult to advance the second catheter 6, an operation of pulling the delivery wire 3 toward the proximal side D1 may be performed in a state where the locking stent 2 is locked to the inner wall V1 of the biological lumen V. By this pulling operation, the second catheter 6 can be advanced to the distal side D2.

Specifically, when the delivery wire 3 is pulled toward the proximal side D1 in a state in which the locking stent 2 is locked to the inner wall V1, the path of the delivery wire 3 in the biological lumen V becomes close to a straight line and becomes short. At this time, when a portion of the proximal side D1 of the second catheter 6 is held directly or indirectly, the position of the portion of the proximal side D1 of the second catheter 6 does not change, and instead, the distal end 61 of the second catheter 6 advances in the biological lumen V. Further, when the distal end 61 of the second catheter 6 advances into the biological lumen V without changing the position of the portion of the second catheter 6 on the proximal side D1, the path of the second catheter 6 becomes close to a straight line. This operation is particularly beneficial when the second catheter 6 attempts to pass through a place where it is difficult to pass through, for example, a place where the bending of the biological lumen V is severe or a place where the inner diameter is small.

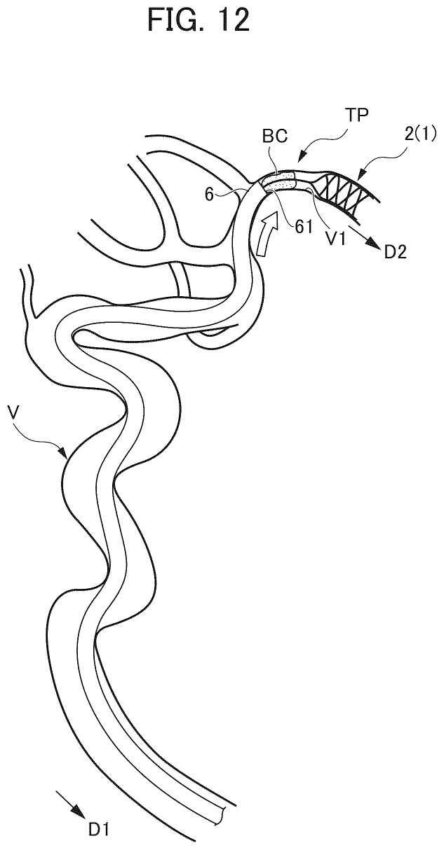

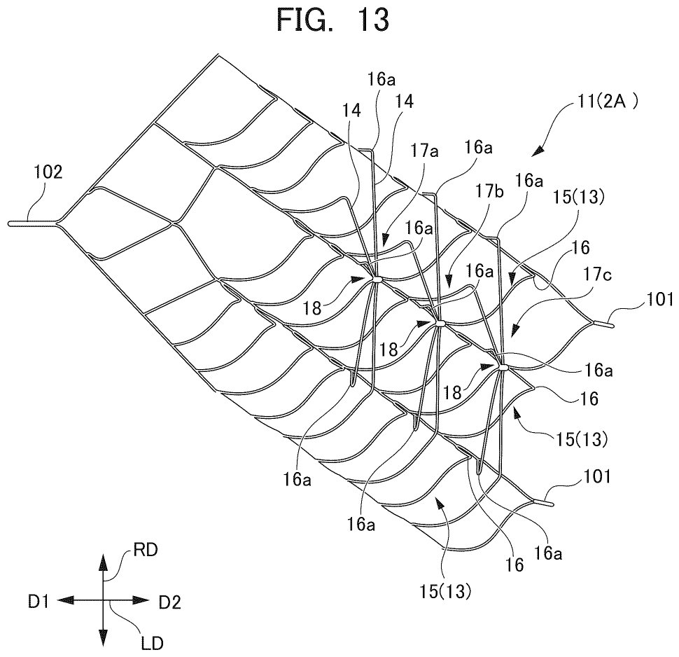

By advancing the distal end 61 of the second catheter 6 within the biological lumen V, the distal end 61 of the second catheter 6 can be brought into contact with the thrombus BC as shown in FIG. 12. Thereafter, the first catheter 5 is removed, suction force is generated in the second catheter 6 (thrombus aspiration catheter), the thrombus BC is drawn to the proximal side while being suctioned to the distal end 61 of the second catheter 6, and the thrombus BC is recovered to the outside of the living body. When the thrombus BC is small or the thrombus BC can be crushed to be small, the thrombus BC may be collected outside the living body by suctioning the thrombus BC into the lumen of the second catheter 6 at a position where the distal end 61 of the second catheter 6 is in contact with the thrombus BC.

Although not shown, after the thrombus BC is collected outside the living body, the first catheter 5 is slidably fitted over the delivery wire 3 in an inserted manner and sent to the proximal side of the locking stent 2, the locking stent 2 is resheathed to the distal end of the first catheter 5, and the locking stent 2 and the delivery wire 3 are recovered together with the first catheter 5. FIGS. 6 to 12 each show an example of the operation of collecting the thrombus by the distal stabilizer 1 and the catheter and an outline thereof. In the operation of collecting the thrombus by the distal stabilizer 1, various surgical operations are performed according to the site where the thrombus has formed in the biological lumen V, the shape and size of the thrombus, and the like.

According to the distal stabilizer 1 of the present embodiment, for example, the following advantageous effects are obtained. The distal stabilizer 1 according to the embodiment includes the filter portions 17 in the locking stent 2 (see FIG. 2). According to this configuration, even when foreign matter such as a thrombus or fragments thereof migrate to the distal side by the operation of delivering the second catheter 6 to the target position, since most of the foreign matter can be collected by the filter portions 17 of the locking stent 2, it is possible to more reliably suppress the scattering of the foreign matter toward the distal side. As described above, since it is possible for the distal stabilizer 1 according to the present embodiment to suppress scattering of the thrombus or the fragments thereof to the distal side, it is possible to reduce the risk of embolization of the biological lumen on the distal side. Such an effect can be expected not only for thrombus and fragments thereof but also for foreign matter such as plaque.

In the distal stabilizer 1 of the embodiment, the plurality of filter portions 17 are provided in the axial direction LD of the locking stent 2 (refer to FIG. 2). Therefore, it is possible to make the thrombus collected by the filter portion 17 of the locking stent 2 more difficult to migrate. In addition, it is possible to collect more thrombus and fragments thereof.

In the distal stabilizer 1 of the embodiment, the struts constituting the respective filter portions 17 are provided at different positions in the circumferential direction (refer to FIG. 3). As a result, when the locking stent 2 is viewed from the axial direction LD, the struts 14 constituting the respective filter portions 17 are substantially uniformly arranged without being extremely biased (refer to FIG. 5D). Therefore, in the filter portions 17 of the locking stent 2, it is possible to collect more thrombus and fragments thereof in a wide range.

In the distal stabilizer 1 of the embodiment, the locking stent 2 has a mesh pattern structure in which a plurality of cells each having a shape surrounded by the struts 14 are spread. The filter portion 17 is formed by the plurality of struts 14 constituting the free protruding end 16 of the cell (open cell) protruding from the outer circumferential side to the center side in the radial direction (refer to FIG. 4). According to this configuration, since it is possible to provide the filter portion 17 by adopting a portion of the cells 13 constituting the locking stent 2, it is possible to provide the filter portion 17 more easily in the locking stent 2.

In the distal stabilizer 1 of the embodiment, the ends of the respective struts 14 constituting the filter portion 17 are coupled to each other to constitute one tip portion 18 (refer to FIG. 4). Therefore, it is possible to suppress a situation in which the thrombus or fragments thereof collected by the locking stent 2 passes through the distal end portion 18 of the filter portion 17 and is scattered to the distal side.

In the distal stabilizer 1 of the embodiment, the free protruding end 16 of the cell constituting the locking stent 2 protrudes to the distal side D2 in the direction of inserting the locking stent 2 into the catheter (refer to FIG. 3). According to this configuration, when the locking stent 2 is accommodated in the catheter, particularly, the first catheter 5 having a small inner diameter after the operation of the catheter delivery, the free protruding end 16 is less likely to interfere with the opening on the distal side of the catheter, such that it is possible to smoothly accommodate the locking stent 2 in the catheter.

Although embodiments of the present invention have been described above, the present invention is not limited to the above-described embodiments, and various modifications and changes such as those described below can be made and are also included in the technical scope of the present invention. In addition, the advantageous effects described in the embodiments merely exemplify the most preferable effects produced from the present invention, and are not limited to those described in the embodiments. Although the above-described embodiments and modifications described below can be appropriately combined and used, detailed descriptions thereof will be omitted.

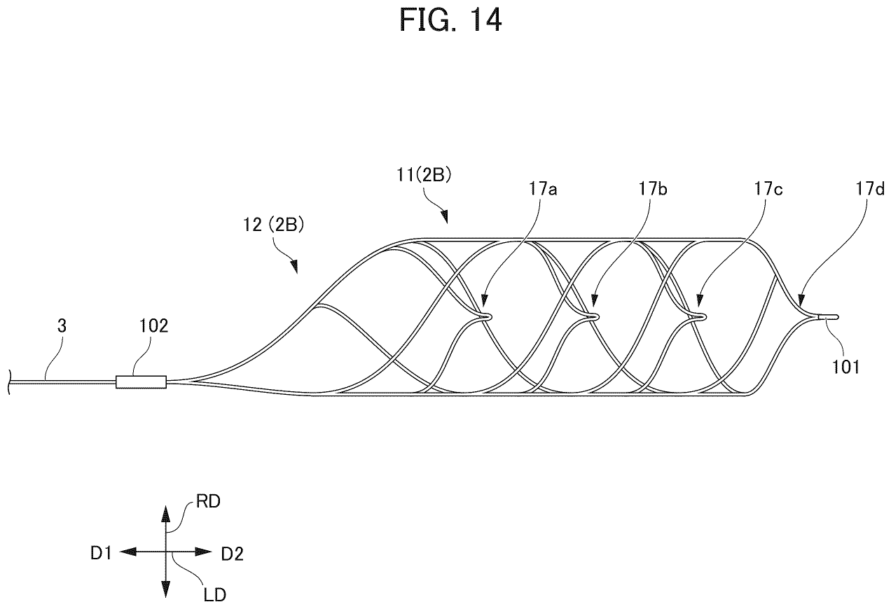

In the following description of the modifications and the drawings, portions having the same functions as those of the above-described embodiments are denoted by the same reference numerals, and redundant descriptions thereof are appropriately omitted. FIG. 13 is an expanded view showing a state in which a locking stent 2A of a modification is virtually expanded in a plane. FIG. 13 corresponds to FIG. 2 of the embodiment. As shown in FIG. 13, the locking stent 2A of the modification is different from the locking stent 2 of the embodiment in that free protruding ends 16a of each of the cells 13 constituting the filter portion 17 face outward in the radial direction RD. According to the present embodiment, since the inner wall is strongly pressed by the free protruding ends 16a facing outward in the radial direction RD, the struts 14 connected to the free protruding ends 16a protrude from the outer circumferential side to the center side in the radial direction, and thus it is possible to suppress a decrease in the locking force of the locking stent 2 to the inner wall. In the modification shown in FIG. 13, the free protruding ends 16 of the other cells 13 which do not constitute the filter portion 17 may also face outward in the radial direction RD.

FIG. 14 is a side view of a locking stent 2B according to modification. FIG. 14 corresponds to FIG. 2 of the embodiment. As shown in FIG. 14, the locking stent 2B of the modification includes a filter portion 17d at the end on the distal side D2. The configuration of the filter portion 17d is the same as that of the filter portions 17a to 17c, and is formed by two struts 14 constituting the free protruding end 16 of each of the cells 13 protruding from the outer circumferential side to the center side in the radial direction RD (see FIG. 4). In the filter portion 17d, the distal marker 101 is provided at a tip portion protruding toward the distal side D2. According to the locking stent 2B of the modification shown in FIG. 14, even when foreign matter such as a thrombus or fragments thereof migrate to the distal side by the operation of delivering the second catheter 6 to the target position, since most of the foreign matter can be collected by the filter portions 17a to 17d of the locking stent 2B, it is possible to more reliably suppress scattering of the foreign matter to the distal side.

In the locking stent 2 according to the embodiment, the number of the filter portions 17 is not limited to three, and one or four or more may be provided. In addition, the filter portions 17a to 17c may be disposed at uneven intervals in the axial direction LD of the locking stent 2.

In the locking stent 2 according to the embodiment, the filter portion 17 may be made of a wire-shaped member (an independent wire-shaped member not constituting cells) coupled to the inner circumferential surface of the main body portion 11. That is, in the locking stent 2, filter portions each formed by the cell 13 having the free protruding end 16 and filter portion each formed by an independent wire-shaped member not constituting the cell may coexist.

In the locking stent 2 according to the embodiment, the free protruding ends 16 (tip portions each connecting the two struts 14) each constituting the tip portion 18 of the filter portion 17 may be configured not to be coupled to each other. For example, the free protruding ends 16 may not be coupled to each other and may be merely in contact with each other, or the free protruding ends 16 may not be in contact with each other.

### EXPLANATION OF REFERENCE NUMERALS

1 distal stabilizer
2 (2A, 2B) locking stent
3 delivery wire
10 delivery system
13 (13a to 13c) cell
14 strut
15 open cell portion
16 (16a) free protruding end
17 (17a to 17c) filter portion
18 tip portion

1. A method comprising delivering to a target position on a distal side in a biological lumen a treatment catheter or a treatment device via the treatment catheter provided at the target position, wherein
the method employs a distal stabilizer as an anchoring device when delivering the treatment catheter to the target position, and
the distal stabilizer includes
a linear delivery member; and
a self-expansion member including an antenna portion and a main body portion that is coupled to a distal end of the linear delivery member and is to be locked in the biological lumen in a state of being expanded in diameter into a cylindrical shape,
wherein the antenna portion is a portion that converges a portion on a proximal side of the main body portion to the linear delivery member, and
wherein the main body portion includes a filter portion constituted by a wire-shaped member that protrudes from an outer circumferential side toward a center side in a radial direction of the main body portion.

2. The method according to claim 1, wherein the filter portion comprises a plurality of filter portions provided in an axial direction of the main body portion.

3. The method according to claim 2, wherein the plurality of filter portions each include a plurality of wire-shaped members that each protrudes from the outer circumferential side toward the center side in the radial direction of the main body portion, and positions of the plurality of wire-shaped members in a circumferential direction are different from one another.

4. The method according to claim 3, wherein, in each of the plurality of filter portions, end portions of the plurality of wire-shaped members that each protrude from the outer circumferential side toward the center side in the radial direction of the main body portion are coupled to each other.

5. The method according to claim 1, wherein the main body portion has a mesh pattern structure in which a plurality of cells each having a shape surrounded by the plurality of wire-shaped members are spread,
at least one of the plurality of cells is an open cell having at least one free protruding end, and
at least one of the plurality of filter portions is provided by the plurality of wire-shaped member constituting the free protruding end of the filter portion protruding from the outer circumferential side toward the center side in the radial direction of the main body portion.

6. The method according to claim 5, wherein the free protruding end of the cell protrudes toward a distal side in a direction of inserting the self-expansion member into a catheter.

7. The method according to claim 1, wherein the self-expansion member is locked on a distal side of a target position in a biological lumen.
